(19) United States
(12) Patent Application Publication
(10) Pub. No.: US 2007/0073370 A1
(43) Pub. Date: Mar. 29, 2007

(54) Trans-septal anchoring system and method

(76) Inventors: Todd M. Zielinski, Minneapolis, MN (US); Douglas A. Hettrick, Blaine, MN (US); Phillip C. Falkner, Minneapolis, MN (US); Kevin R. Seifert, Forest Lake, MN (US); Vicki L. Bjorklund, Maple Grove, MN (US); Mark D. Schneider, Mound, MN (US)

Correspondence Address: MEDTRONIC, INC., 710 MEDTRONIC PARK, MINNEAPOLIS, MN, 55432-9924, US

(21) Appl. No.: 11/235,942

(22) Filed: Sep. 27, 2005

(51) Int. Cl. A61N 1/05

(52) U.S. Cl. 607/119

(57) ABSTRACT

A pressure sensor, in one embodiment, is passed through the atrial septal wall. A plurality of anchors is disposed on each side of the septal wall and secures the position of the pressure sensor. An inflatable deployment balloon is used to actuate the anchors.

### BACKGROUND OF THE INVENTION

1. Field of the Invention

The present invention relates to implantable medical devices. More specifically, the present invention relates to implantable medical devices that sense or measure a cardiac parameter.

2. Description of the Related Art

There are a number of implantable medical devices (IMDs) that sense various physiological parameters and/or provide a variety of therapies. For example, implantable pulse generators (IPGs) typically include one or more leads that are in contact with cardiac tissue to sense electrical depolarization and provide pacing stimuli. Implantable cardioverter/defibrillators (ICDs) also typically include one or more leads and provide a larger stimulus for cardioversion or to defibrillate the heart. Often, IMDs include both pacing and cardioversion/defibrillation capabilities.

A housing containing the pulse generator, battery, capacitors, processor, memory, circuitry, etc. is implanted subcutaneously. One or more leads are delivered transvenously such that electrodes forming a portion of the lead are disposed within or contacting an outer portion of the heart. The housing, or “can,” may also include one or more electrodes that are selectively used in combination with the various lead electrodes.

In general, the leads sense electrical activity of the heart, typically represented as an electrogram (EGM), which is indicative of the cardiac depolarization waveform and indicates the timing of the various components of the complex. This data indicates whether and when intrinsic events occur, their duration and morphology. The timing of certain events (or their failure to occur when expected) is used to trigger various device actions. For example, sensing an atrial depolarization may begin a timer (an escape interval) that leads to a ventricular pacing pulse upon expiration. In this manner, the ventricular pacing pulse is coordinated with respect to the atrial event.

The heart includes four chambers; specifically a right and a left atrium and a right and left ventricle. Leads are commonly and routinely placed into the right atrium as well as the right ventricle. For left-sided applications, the lead is typically guided through the coronary sinus and into a cardiac vein. One or more electrodes are then positioned (within the vein) to contact an outer wall of the left atrium and/or left ventricle. While direct access to the interior of the left atrium and left ventricle is possible, it has been historically less preferable. As the left ventricle provides oxygenated blood throughout the body, a foreign object disposed on the left side and providing a sufficient obstruction could lead to the formation of clots and would increase the risk that such a clot would form and be dispersed.

The sensing and utilization of electrical data is commonly employed as the electrodes used for delivering stimulus are typically also useful in sensing this data. This is generally non-problematic in left-sided applications, as the electrical waveform is adequately sensed from the above-described left side lead placement position.

A wide variety of other sensors are employed to sense parameters in and around the heart. For example, flow rates, oxygenation, temperature and pressure are examples of parameters that provide useful data in certain applications. Obtaining such data on the right side is typically non-problematic; however, obtaining the same data directly from the left side is made more difficult by the above-noted desire to minimize invasiveness into the left atrium or ventricle.

Pressure data, in particular, is a useful parameter in determining the presence, status and progression of heart failure. Heart failure often leads to an enlargement of the heart, disproportionately affecting the left side in many cases. Left side pressure values would be useful in monitoring the patient's condition; gauging the effectiveness of a given therapy such as Cardiac Resynchronization Therapy (CRT); and timing, controlling or modifying various therapies.

Left atrial pressure, in particular, is one variable that defines the status of heart failure in a patient. Attempts have been made to measure surrogates of this variable by monitoring pulmonary wedge pressure in clinical care. Measurement of ePAD with implantable devices such as the Medtronic Chronicle™ have been used to measure real-time intracardiac chamber pressure in the right ventricle and provide an estimate of mean left-sided pressure. These techniques generally do not provide certain phasic information and do not necessarily correlate with left atrial pressures under certain conditions such as pulmonary hypertension or intense levels of exercise.

### BRIEF DESCRIPTION OF THE DRAWINGS

FIG. 1 illustrates an implantable medical device (IMD) having a plurality of leads implanted within a heart.

FIG. 2 is a block diagram illustrating the functional components of an IMD.

FIG. 3 is an illustration of a heart showing an interior view of a right atrium and indicating the location of the fossa ovalis.

FIG. 4 is a schematic illustration of a pressure sensor coupled with a medical lead.

FIG. 5 is a schematic illustration of the pressure sensor and lead with a sheath having deployable anchors.

FIG. 6 is a schematic diagram of a delivery catheter.

FIGS. 7-13 illustrate the lead and sheath in various stages of deployment.

FIG. 14 is a schematic end, sectional view of a plurality of deployed anchors.

FIG. 15 is a schematic illustration of a lead with a pressure sensor and a sheath having deployable anchors and a deployment balloon.

FIGS. 16-23 illustrate the lead of FIG. 15 in various stages of deployment.

### DETAILED DESCRIPTION

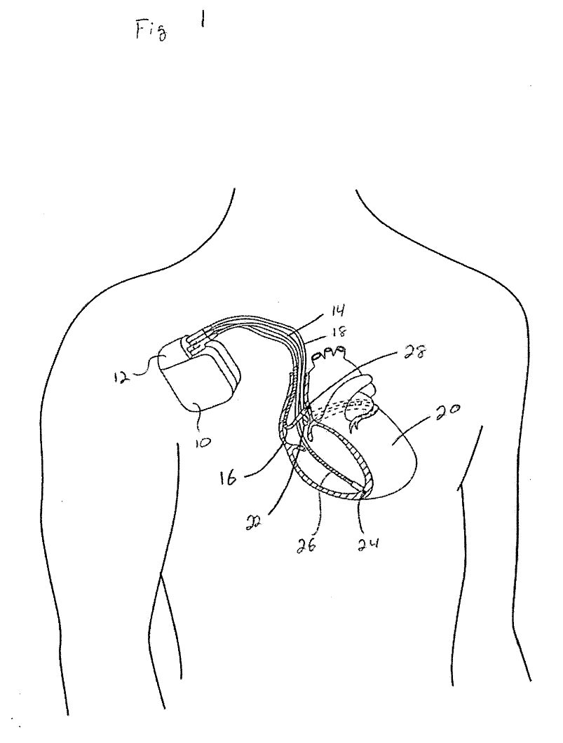

FIG. 1 illustrates an implantable medical device (IMD) 10 that includes pacing, cardioversion and defibrillation capabilities. A header block 12 forms a portion of the IMD 10 and three leads 14, 16, 18 are illustrated as coupled with the header block. A right ventricular lead 14 is disposed in the right ventricle of the heart 20. More specifically, a helical electrode tip 24 is embedded into the apex of the right ventricle. The electrode tip 24 forms or is part of a tip electrode and a coil electrode 26 is also included. A ring electrode may be disposed between the tip electrode 24 and the coil electrode 26.

An atrial lead 16 is disposed within the right atrium such that an electrode 28 contacts an interior wall of the right atrium. A left-sided lead 18 is illustrated as passing through the coronary sinus 22 and into a cardiac vein. In this position, the left-sided lead 18 has a distal end in contact with an outer wall of the left ventricle. The IMD 10 includes a housing that can act as an electrode or, though not illustrated, may include multiple electrodes. With such a configuration, pacing stimuli is selectively delivered to the right atrium, the right ventricle, and/or the left ventricle. Likewise, a defibrillation pulse may be generated from any given electrode to any second electrode, such that the defibrillation waveform traverses the desired portion of the heart 20.

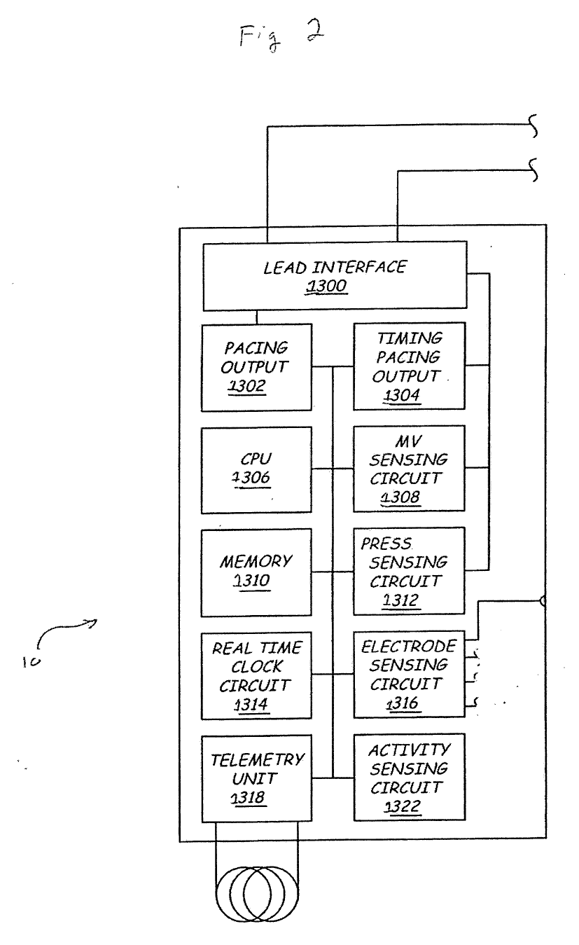

FIG. 2 is a simplified schematic diagram illustrating certain components of the IMD 10. The IMD 10 includes a processor or CPU 1306, memory 1310, timing circuits 1314, timing output circuit 1304, pacing and defibrillation output circuits 1302, an appropriate lead interface 1300, and appropriate electrode sensing circuits 1316. The operation of the IMD 10 may be controlled by software or firmware and may be reprogrammed and/or provide data to an external device via telemetry unit 1318.

Also illustrated are exemplary sensing units that may be included with IMD 10. For example, an activity sensing circuit 1322, and a minute ventilation circuit 1308 are included. Thus far, IMD 10 is illustrated in an exemplary manner, may or may not include all components illustrated, and may include many additional components and capabilities without departing from the spirit and scope of the present invention.

A pressure sensing circuit 1312 receives input from the pressure sensor described herein. In one embodiment, a pressure sensor is included on the right atrial lead 16 or a similar structure deployed within the right atrium. The pressure data, when received, is used by the CPU 1306 to monitor or control therapy, monitor the status of the heart, and/or to provide information to an external device via telemetry unit 1318. It should also be appreciated that various pressure sensors may provide relative data and an absolute pressure sensor (not shown) may be positioned external to the heart and utilized to provide reference data via telemetry unit 18 and/or to the external device.

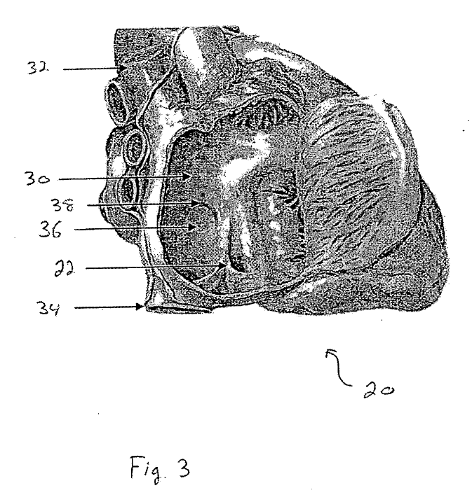

FIG. 3 is an illustration of the anatomy of a human heart 20. In particular, the interior of right atrium 30 is illustrated, along with the superior vena cava 32 and inferior vena cava 34. The atrial septum, dividing the right atrium from the left atrium, is primarily defined (from the right-side perspective) by the fossa ovalis 36. Surrounding the fossa ovalis 36 is the fossa limbus 38, which is a raised muscular rim. The fossa ovalis 36 is a relatively thin, but very strong membrane that separates the right atrium from the left atrium and is a non-conductive pathway for depolarization. The fossa ovalis 36 marks the previous location of the foramen ovale, which in embryonic and fetal development provided for direct passage between the atrial chambers. The fossa limbus 38 and the atrial tissue surrounding the fossa limbus 38 is conductive.

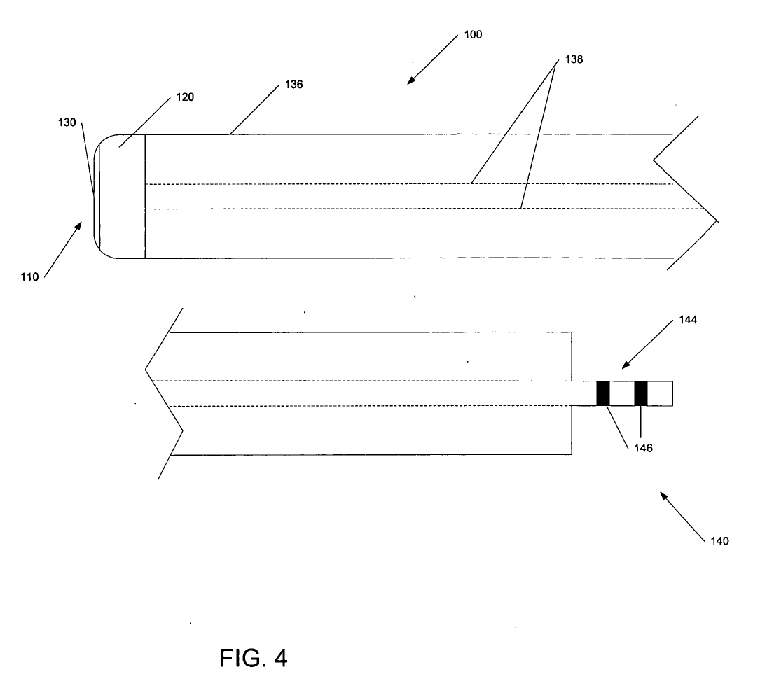

FIG. 4 is a schematic view of a portion of a lead 100 that includes a pressure sensor 120 disposed at a distal end 110 of a lead body 136. The pressure sensor 120 includes a transducing membrane 130 primarily located within a plane perpendicular to the main axis of the lead 100. A pair of conductors 138 is schematically illustrated as electrically coupling the pressure sensor 120 to a connector pin 144 disposed at a proximal end of the lead body 136. The header block 12 receives the connector pin 144 and is electrically coupled with the conductors 138 via the contacts 146. This illustrates one embodiment wherein data is communicated over one or more conductors from the pressure sensor 120 to the IMD 10. Of course, various other arrangements are contemplated for the exchange of data with, and the delivery of power to, the pressure sensor 120, all of which are within the scope of the present invention. Lead body 136 is sufficiently flexible to permit transvenous implantation, while retaining integrity.

Intracardiac pressure sensing may be accomplished in a number of ways. The following U.S. patents disclose a variety of pressure sensors and are herein incorporated by reference in their entireties: U.S. Pat. Nos. 6,223,081; 6,221,024; 6,171,252; 6,152,885; 5,919,221; 5,843,135; 5,368,040; 5,353,800; and 4,967,755. In the illustrated example, pressure transducer membrane 130 is a high fidelity pressure transducer configured for placement within the left atrium. Various other positional arrangements may be utilized without departing from the scope of the present invention. The present invention may also be employed to deliver a pressure sensor 120 into the left ventricle through the ventricular septal wall from the right ventricle. Mechanically, the present invention will operate in the same manner as described herein with appropriate dimensional changes. The ventricular septal wall is thicker than the atrial septal wall 220 and makes passage therethrough more difficult. The process is further complicated by the location of the Bundle of His, which, if intact, is preferably avoided during the implantation process. The present invention would also provide a mechanism for His bundle pacing. Thus, while the embodiments are described with respect to atrial placement, the invention is not so limited and includes placement and use within the ventricles.

Phasic information of the left atrial pressure provided by the pressure sensor 120 can be used, for example, by the IMD 10 to control several pacing parameters such as AV timing and VV timing for management of AF and CHF by optimizing left-sided filling and ejection cycles and enhancing cardiovascular hemodynamic performance. Such data may also be used for assessment of mitral regurgitation and stenosis. For device-based management of atrial fibrillation, the phasic information can be used for discriminating atrial fibrillation from flutter and optimizing atrial anti-tachycardia pacing therapies.

Pressure sensor 120 provides diagnostic data to clinicians and/or control device operation by automated feedback control. Direct, real-time left atrial pressure measurement may be utilized to provide diagnostic information for management of heart failure and in patients with pacemakers, to optimize pacing parameters in order to prevent its progression. In addition, pressure sensor 120 provides information about the atrial substrate for management of AF and may control pacing parameters to prevent progression of AF. Reference is made to U.S. patent application Ser. No. 11/097,408, filed on Mar. 31, 2005, and titled “System and Method for Controlling Implantable Medical Device Parameters in Response to Atrial Pressure Attributes,” which is herein incorporated by reference in its entirety.

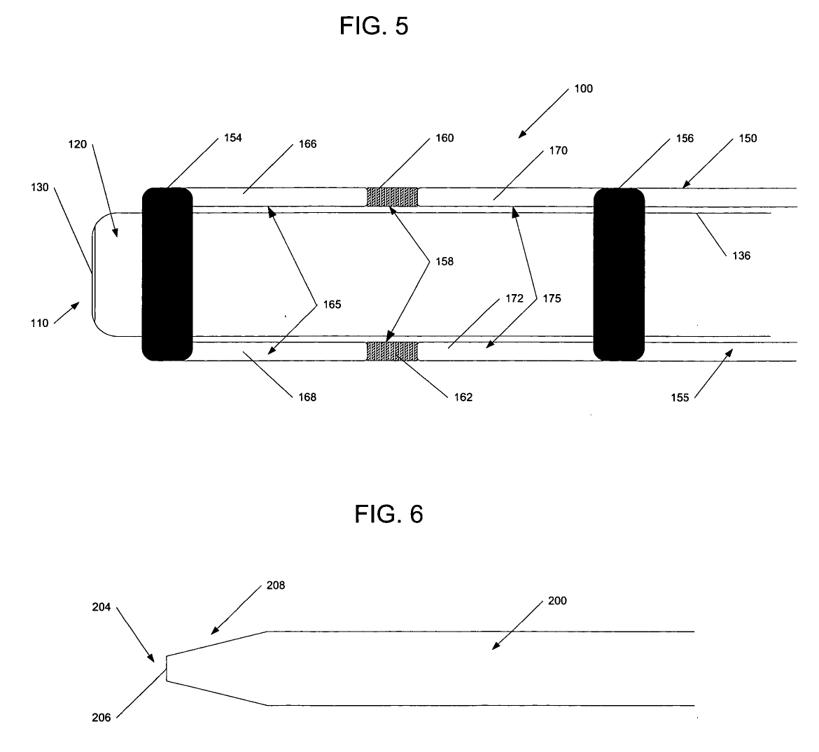

FIG. 5 is a schematic illustration of lead 100. A sheath 150 is provided. In one embodiment, the sheath 150 is fabricated from an appropriate biocompatible material, such as urethane. The lead body 136 is disposed within the sheath 150 and is moveable relative to the sheath 150. A pair of radio-opaque rings 154, 156 provides a mechanism to identify a specific, known location during the implantation process by being readily visible during fluoroscopy or an appropriate image-guiding technology. The rings 154, 156 may be fabricated from an alloy such as platinum/iridium. In the present embodiment, these rings provide various mechanical functions that will be described; however, it should be appreciated that this functionality may be separated from the imaging characteristics, and that more or fewer image-identification mechanisms may be provided.

Distal ring 154 is fixed with respect to the lead body 136, and hence with respect to the pressure sensor 120. Proximal ring 156 surrounds lead body 136 but is not fixed; rather, proximal ring 156 may be moved axially in a proximal or distal direction (as illustrated) with respect to lead body 136.

The sheath 150 has four sections. A proximal sheath section 155 extends from a proximal side of the ring 156 over a majority of the lead body 136 towards the proximal end. The proximal sheath section 155 is fixedly coupled with the proximal ring 156 so that actuation of the proximal sheath section 155 will cause the proximal ring 156 to slide in either a proximal or distal direction, or rotate accordingly. Pivotably coupled to a distal side of the proximal ring 156 are one or more interior anchors 175. In this view, two interior anchors 170,172 are illustrated. In the current embodiment, the interior anchors 175 are fabricated from the same material as the proximal sheath section 155; though this is not required. Similarly, one or more exterior anchors 165 are pivotably coupled to a proximal side of the distal ring 154. In this embodiment, two exterior anchors 166, 168 are illustrated. The terms “interior” and “exterior” are used to facilitate the description and provide an indication of which ring 154, 156 a given anchor is pivotably attached to; no further limitation of any kind is meant or implied by such terms. When implanted, the interior anchors 170 will remain in the initial cardiac chamber, e.g., the right atrium 30, whereas the exterior anchors will be located within the secondary cardiac chamber; that is, the chamber the sensor is deployed into, e.g., the left atrium.

Prior to implantation, the interior anchors 175 are coupled with the exterior anchors 165; each at a respective break point 158. Break points 160, 162 are illustrated. The break points 158 initially maintain the sheath 150 as an integral unit prior to and during a portion of the implantation. When the proximal ring 156 is advanced relative to the lead body 136 in the distal direction, towards the distal ring 154, the break points 158 act as flex points or flexible joints, as will be described in greater detail below. Finally, the break points 158 sever the connection between their respective interior and exterior anchors 175, 165. In one embodiment, the break points 158 are formed from a biocompatible, biodegradable material that breaks down in a controllable or known manner when exposed to bodily fluids, such as blood. For example, the break points 158 may be formed from a gelatinous material or a sugar composite.

In alternative embodiments, the break point 158 is configured so that flexing of the break point 158 causes it to sever, either when flexed to a predetermined angle, by repeatedly flexing the joint, or a combination of the two. Similarly, this separation may be accomplished via rotation of the proximal sheath section 155 relative to the lead body 136. As mentioned, the distal ring 154 is fixed in position relative to the lead body 136; this fixation could either permit or preclude rotational movement of the distal ring 154 relative to the lead body 136. If precluded, the rotation of the proximal sheath portion 155, while retaining the lead body 136 in a static position, will impart torque to the break points 158. This may lead to their forcible separation, e.g., along a predefined score line; or the anchors 165, 175 could be coupled by a sliding hinge or lip member which separates upon sufficient rotation. As yet another alternative, various mechanical separation mechanisms may be utilized. For example, the break point 158 may be formed from a metal or alloy and having a coil configuration; thus, flexibility is provided as the proximal ring 156 is advanced. The coiled break point 158 could then be retracted from a proximal end of the lead 100 on a temporary basis via, e.g., an attached guidewire, thereby allowing the exterior anchors to pivot away. The break points 158 would then be released and form a portion of the interior anchors 170. A similar deployment could occur, leaving the break points coupled with the exterior anchors 165.

Alternatively, the break points could be retracted further along the sheath 150, or removed in their entirety. This may be accomplished by sliding the break points or utilizing a rotational motion to effect longitudinal movement. Depending upon the configuration of the sheath 150, this may occur by movement over an exterior portion of the sheath 150, or within channels or lumens in the sheath 150 provided for this purpose.

As explained in greater detail, the anchors in some embodiments are (or become) independent structures that pivot. This separation of components could extend along the entirety of the proximal sheath portion 155, or there is a transition at the proximal ring from a continuous sheath portion 155 to the anchor section, which includes slots, slits or gaps to define the various independent anchors.

FIG. 6 is a schematic diagram illustrating a delivery catheter 200. The delivery catheter 200 includes a distal tip 204 having a distal opening 206, through which the lead 100 is passed. The distal tip 204 includes a tapered section 208 to facilitate passage through various anatomical structures, including veins, arteries, and valves, as well as orifices created within tissue. Various catheter styles and shapes may be employed without departing from teachings of the present invention.

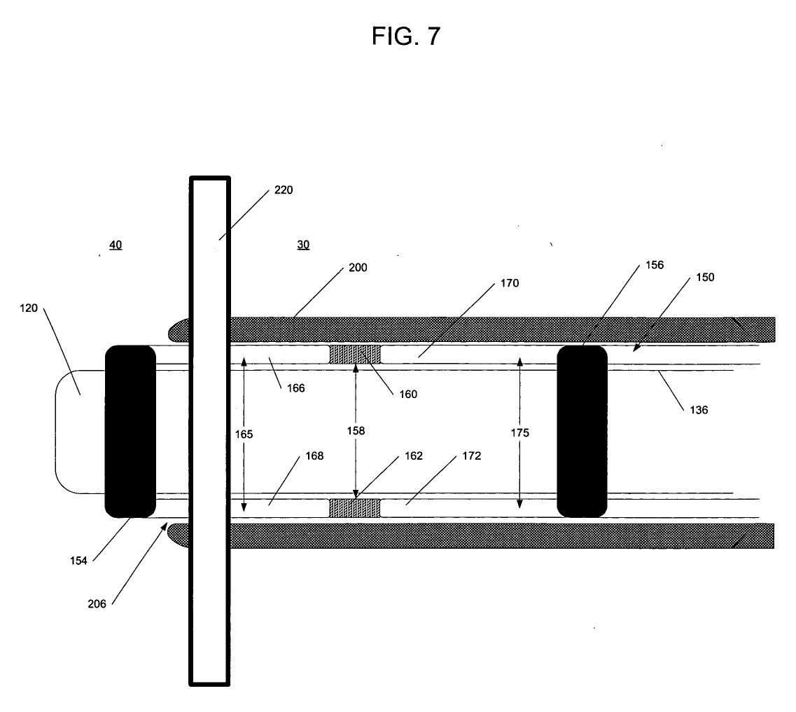

FIG. 7 is a schematic diagram illustrating the lead 100 deployed through the distal opening 206 of the delivery catheter 200, which is passing through an opening in the atrial septal wall 220. This opening in the atrial septal wall 220 is made surgically, utilizing any appropriate technique. In one embodiment, this opening is created in the fossa ovalis 36. In summary, the opening is created and the delivery catheter 200 is deployed through this opening from the right atrium 30 into the left atrium 40. The lead 100 is delivered through the catheter 200 to the position illustrated. The radio-opaque rings 154, 156 are readily identified using the selected imaging technique, such as fluoroscopy. It should be appreciated that, throughout the present application, such techniques are available to determine and confirm position and will not be restated for every instance.

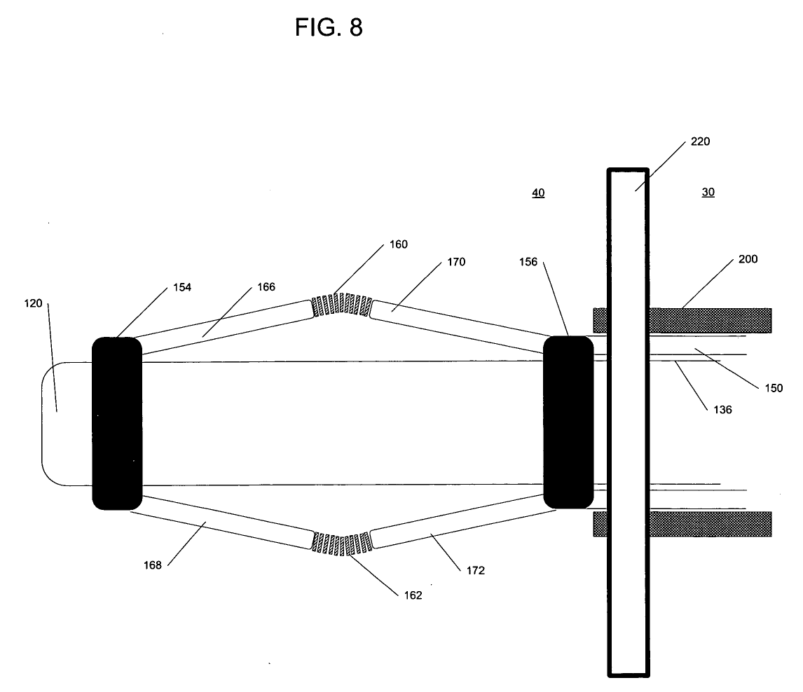

In FIG. 8, the delivery catheter remains relatively fixed (with respect to the position illustrated in FIG. 7) and the lead 100 is advanced further into the left atrium 40. While securing or retaining the lead body 136, the sheath 150 is advanced so that proximal ring 156 moves distally towards distal ring 154. This movement causes the break points 158 to flex. In the present embodiment, the rings 154, 156 are brought sufficiently close that a crease is formed in the break points 158. This alone may cause the break points 158 to separate along this crease; if not, the proximal ring 156 is slid back and forth, repeatedly flexing the break points 158 until they separate. During this process, the interior anchors 175 and exterior anchors 165 pivot at their respective coupling to rings 154, 156. In one embodiment, this forms a bend line in the anchor at the junction which, upon separation of the break point, causes the anchors to have some bias away from the lead body 136.

Separation by flexation may define the interior and exterior anchors 175, 165 in their entirety. That is, the remainder of the break point 158 (after separation) attached to a given anchor is retained and forms part of that anchor. In such an embodiment, the break point 158 may be formed from the same or similar material as the remainder of sheath 150 and provided with a score line, manufactured weakness, and/or manufactured strength/support adjacent to an intended crease line so that flexation occurs in an expected location.

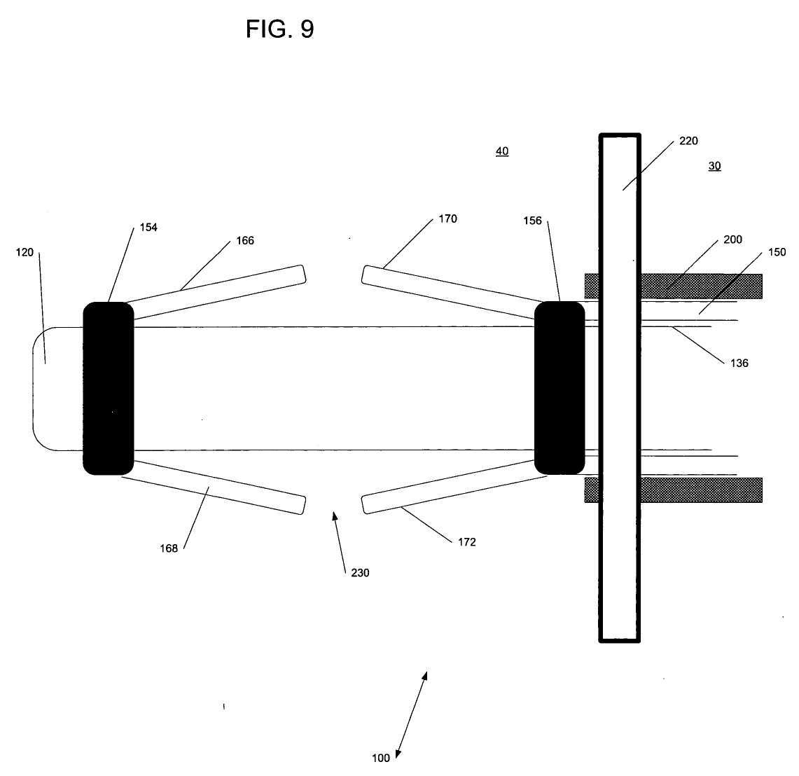

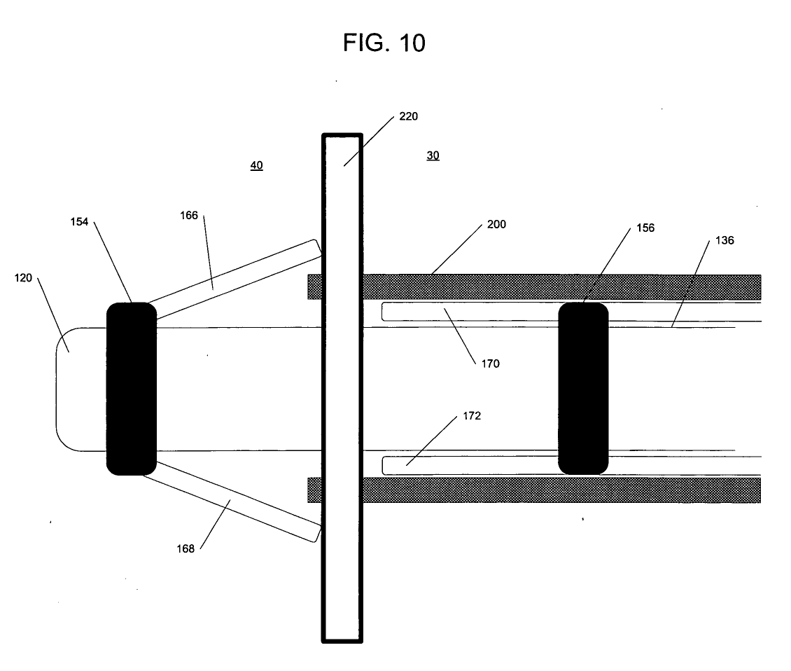

In the present embodiment, the break points 158 are formed from a biocompatible, biodegradable material. After a period of exposure to bodily fluids (e.g., blood), the break points 158 dissolve, and the interior/exterior anchors 175, 165 remain, as illustrated in FIG. 9. In this embodiment, the break points 158 provide the appropriate structural integrity for implantation; however, when dissolved, a well-defined and predictable anchor structure remains. That is, the creasing and separation could lead to uneven structure that may be sharp, jagged or have other unintended structure. During implantation, the dissolving of the break points 158 could be relied upon to separate the interior anchors 175 from the exteriors anchors 165. This would preclude the need to flex the break points 158. The break points 158 would simply need to be exposed to the fluid environment for an appropriate length of time and separation would occur. Naturally, this would delay the remainder of the implantation procedure for a predetermined period of time, whereas flexation allows for a relatively fast separation.

In another aspect of this embodiment, the ability to dissolve the break point 158 based upon time exposure to the fluid environment would permit separation if flexation fails to separate one or more anchors, without requiring the removal of the lead 100. For example, if a given lead 100 had a manufacturing abnormality that precluded the separation by flexation of any anchor, the implanting physician could choose to withdraw the lead 100 through the catheter 200 and replace it with another. Alternatively, that same implanting physician could choose to leave the lead 100 in place and wait for the break points to dissolve, either entirely or until flexation becomes effective. In another scenario, if flexation separates at least one but not all of the anchors, then retraction through the catheter 200 is hindered, if not precluded, by the separated exterior anchors 165 that are at least partially biased away from lead body 136. In this scenario, exposure to the fluid environment will again obviate the problem and separate the remaining anchors.

Returning to FIG. 9, the interior anchors 175 have been separated from the exterior anchors 165. While schematically illustrated, it should be appreciated that the rings 154, 156 may actually be relatively close together, with a correspondingly greater angle between the anchors and the lead body 136 at the junction between the anchors and the rings 154, 156 (as compared to what is illustrated). At this point, the sheath 150 is retracted relative to the lead body 136 so that a gap 230 is defined between the exterior anchors 165 and the interior anchors 175. The minimal size of this gap 230 is such that the interior anchors 175 may be retracted into the sheath 150 without being biased outward by the exterior anchors. To that end, the gap 230 may be zero or even negative, so long as the exterior anchors 165 are not disposed between the interior anchors 175 and the lead body 136. Alternatively, the catheter distal opening 206 of the catheter 200 is selected to be large enough (or resilient enough to expand) so that the interior anchors 175 are retractable even if the exterior anchors 165 are so positioned.

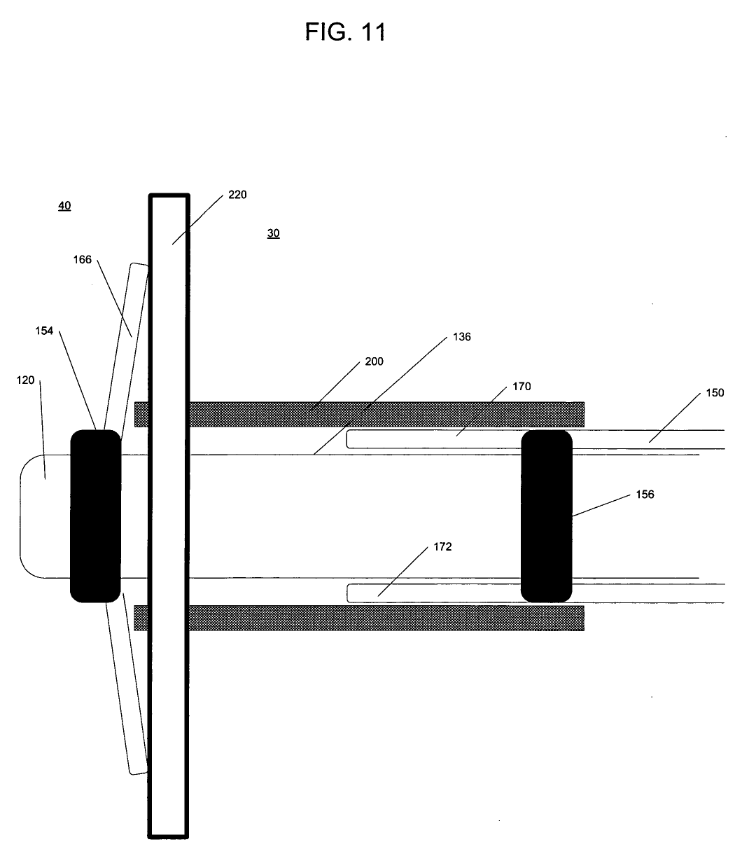

In any event, the lead 100 is retracted into the catheter 200 as illustrated in FIG. 10. The exterior anchors 165 abut the septal wall 220 and pivot outward (away from the lead body 136). The entire lead 100 or the sheath 150 (which is no longer directly coupled with the exterior anchors 165) is retracted until the distal ends of the interior anchors 175 are clear of the septal wall 200. These positions may be confirmed by identifying the location of the radio-opaque rings 154, 156. With further retraction, the exterior anchors 165 are expanded to a fully extended position, as illustrated in FIG. 11. It should be appreciated that the fully-extended position will vary based upon the actual configuration; thus, the angle imparted may vary from that illustrated and still be fully extended. Further, full extension may be, in some embodiments, relative to the position of the pressure sensor 120 to the septal wall 220. That is, to the extent minimal protrusion into the left atrium 40 is desired, the pressure sensor 120 is held as closely as possible to the septal wall 220, which will define the angle of the exterior anchors 165.

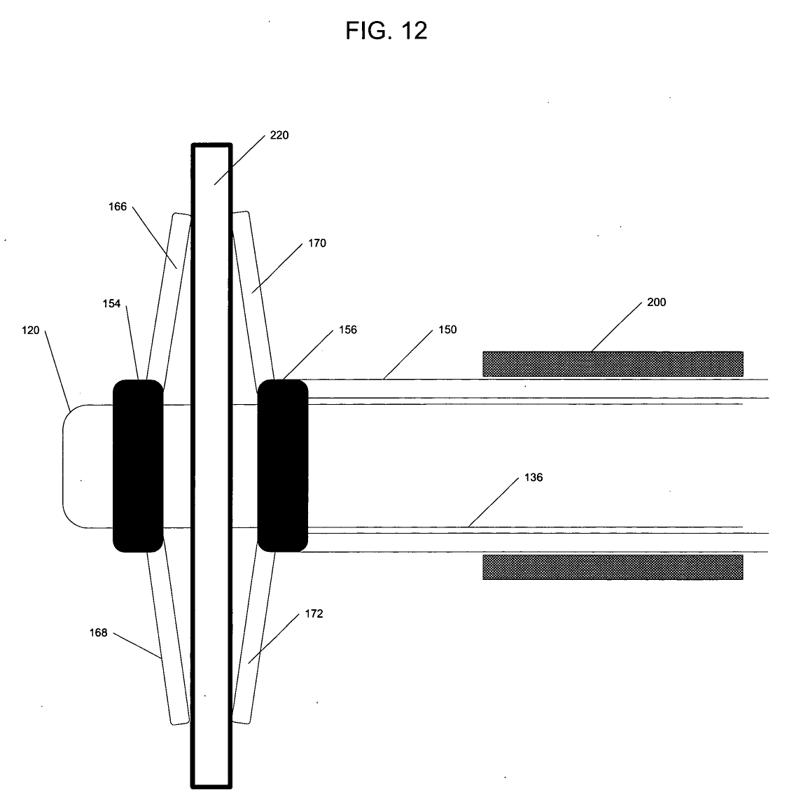

As illustrated in FIG. 12, the sheath 150 is retracted to expose the interior anchors 175 within the right atrium 30. The sheath 150 is advanced in the distal direction, moving the proximal ring 156 towards the distal ring 154. This causes the interior anchors 175 to expand to their fully-extended position, as illustrated.

With the anchors 165, 175 extended, they retain the lead 100 in the position illustrated, relative to the septal wall 220. More specifically, they retain the pressure sensor 120 in the illustrated position within the left atrium 40. That is, the opening created through the septal wall 220 is smaller than the diameter defined by the extended anchors 165, 175 which prevents movement from one atrial chamber to another. Of course, some minor movement may occur due to flexing of the anchors; however, the anchors “sandwich” the septal wall 220, thereby securing the sensor 120 in place. Tissue growth about the anchors 165, 175; the rings 154, 156; the sensor 120; or various other components of the lead 100 will further secure the lead 100 in position.

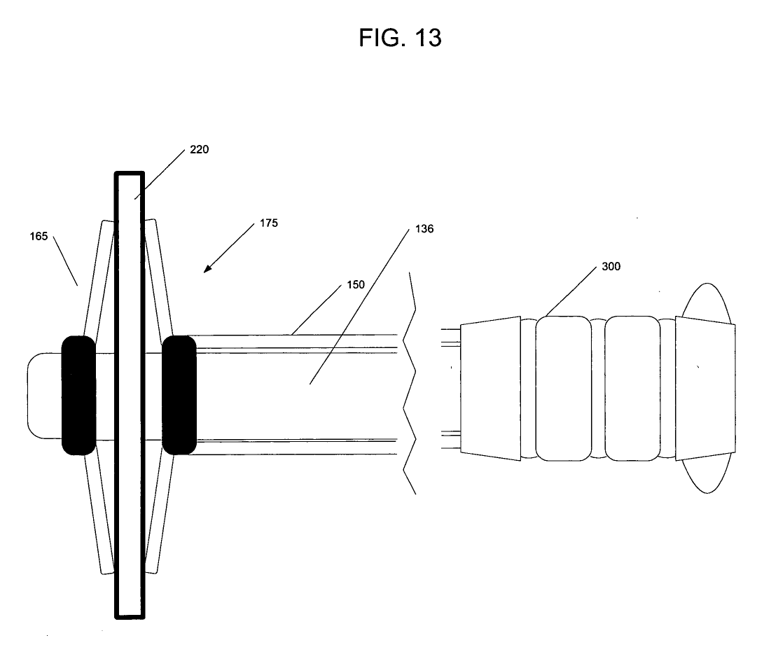

As previously indicated, the distal ring 154 is fixed with respect to the lead body 136, while the proximal ring 156 is moveable relative to the lead body 136. FIG. 13 illustrates an anchor sleeve 300, located at or near the proximal end of lead 100. The anchor sleeve 300 permits manipulation of the proximal ring 156, and hence the sheath 150 relative to the lead body 136. The anchor sleeve 300 also provides a locking function so that the proximal ring 156 and/or the sheath 150 are selectively precluded from moving relative to the lead body 136. After the interior anchors are full expanded (e.g., FIG. 11), the anchor sleeve 300 is used to lock the position. The anchor sleeve 300 may be an element that remains in position such that a portion of the lead 100 and the connector 140 extend from a proximal end of the anchor sleeve 300 so that the lead 100 is coupleable with the IMD 10. Alternatively, the anchor sleeve 300 is utilized to manipulate and/or lock components, and then all or a portion of the anchor sleeve 300 is removed.

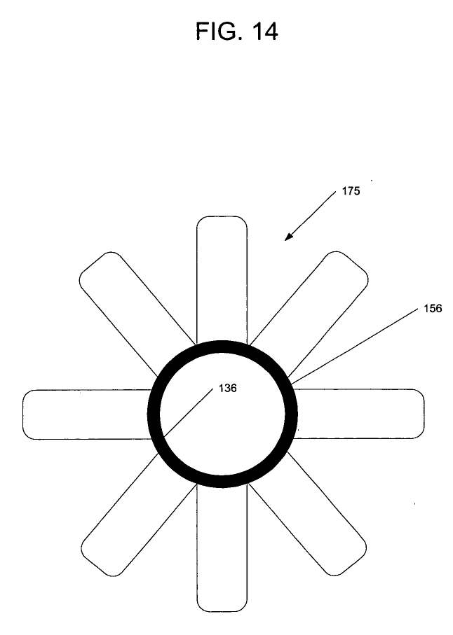

FIG. 14 is an end sectional view, illustrating the anchors 175 in a deployed position. As illustrated, there is a plurality of anchors 175 that extend radially from the proximal ring 156. More or fewer anchors 175 may be utilized. The size and relative proportions of a given anchor 175 are not limited to the embodiment illustrated.

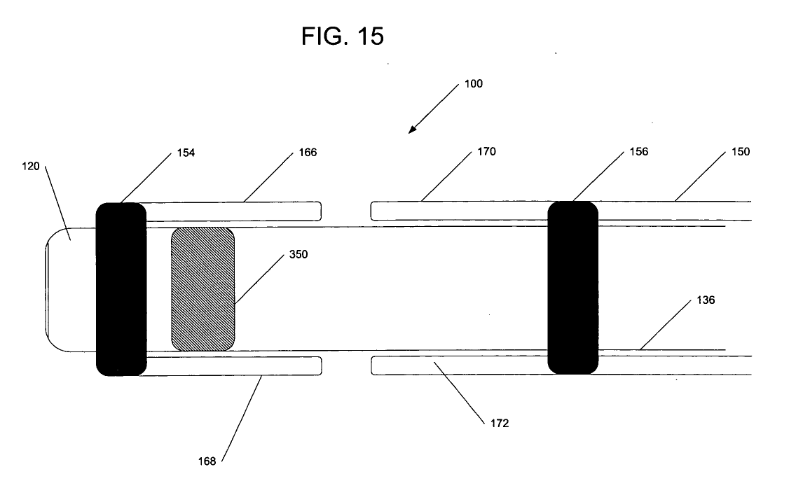

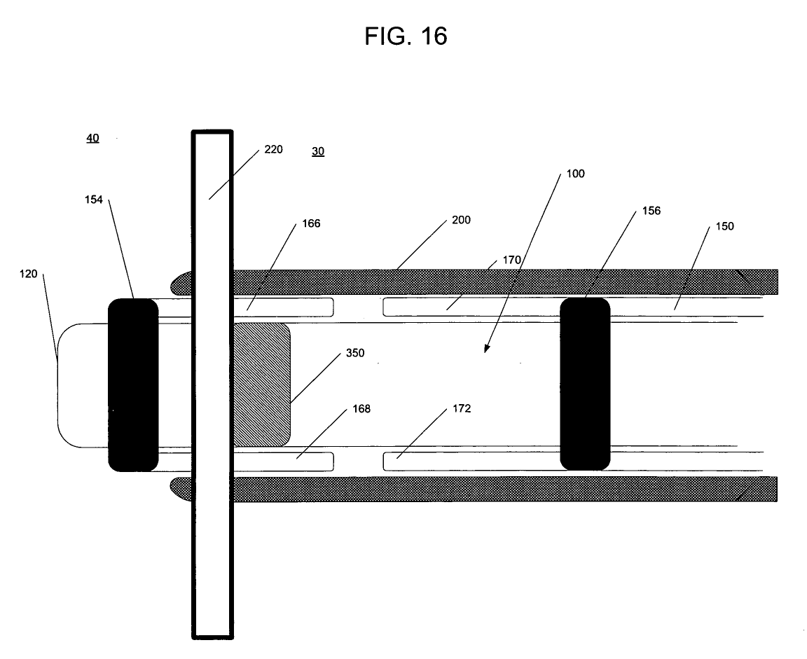

FIG. 15 illustrates an embodiment similar to that of FIG. 5, with like components having the same reference numerals. In the present embodiment, there are no break points 158 (FIG. 5); rather, the exterior anchors 165 are initially separate from the interior anchors 175. A deployment balloon 350 is provided and, in this embodiment, forms a portion of the lead body 136. Though not separately shown, a lumen within the lead body 136 couples the deployment balloon 350 to a proximal access so that the deployment balloon may be selectively inflated or deflated. For example, a syringe may be used to force or compress air through the lumen and cause the deployment balloon 350 to expand. Conversely, the syringe is retracted or the lumen is otherwise opened and the deployment balloon is deflated. In some embodiments, the deployment balloon includes radio-opaque markers that facilitate a visual determination of the location and/or the amount of expansion of the deployment balloon 350. Alternatively, or in addition thereto, by controlling the amount of air deployed by the syringe, the expansion of the deployment balloon may be calculated.

While illustrated as forming a portion of the lead body 136, it should be appreciated that the deployment balloon 350 may be a separate structure from the lead body 136. The lumen may still be disposed within the lead body, or may be external to the lead body 136.

FIG. 16 illustrates the lead 100 partially deployed from within the catheter 200 so that the pressure sensor 120 is within the left atrium 40. The lead body 136 is advanced within the catheter 200 so that the exterior anchors 165 pass through the septal wall 220 and are entirely within the left atrium 40. In the embodiment of FIG. 5, the interior anchors 175 were initially advanced entirely into the left atrium 40 to facilitate the separation at the break point 158. In the present embodiment, this is unnecessary; however, such advancement is not detrimental to the deployment procedure.

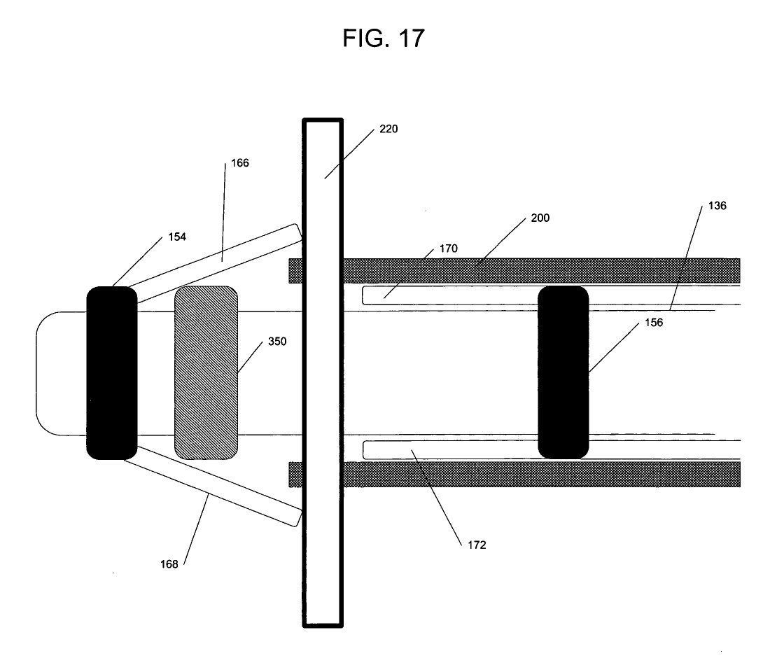

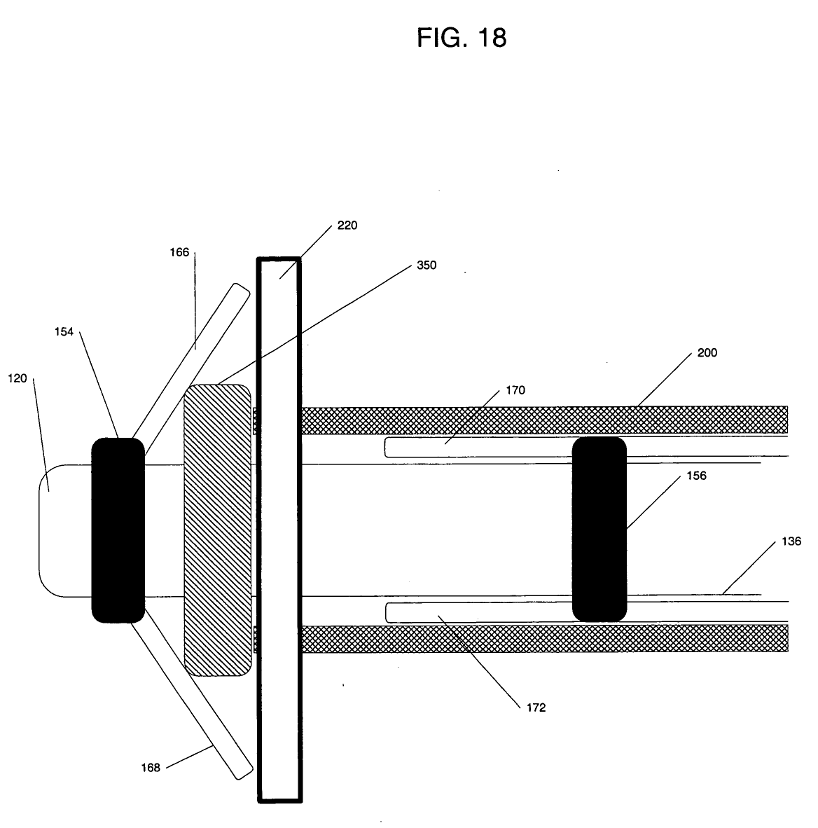

Once at least the exterior anchors 165 entirely pass through the septal wall 220 and the catheter 200, the deployment balloon 350 is inflated so that it expands outwardly from the lead body 136. As this expansion occurs, the deployment balloon 350 contacts the exterior anchors 165 and causes the exterior anchors to pivot or flex at their connection to the distal ring 154, as illustrated in FIG. 17. With continued expansion of the deployment balloon 350, the exterior anchors 165 are likewise further deployed, as illustrated in FIG. 18.

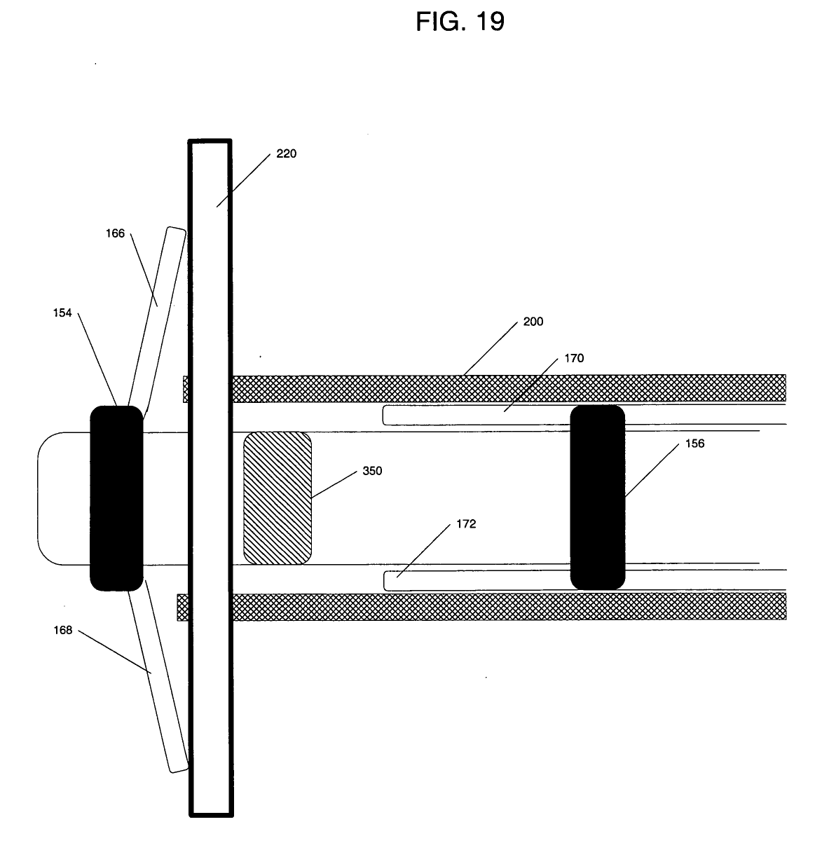

When the exterior anchors 165 are at least sufficiently deployed, the deployment balloon 350 is deflated, at least to a point where the deployment balloon 350 can be retracted into the catheter 200, as illustrated in FIG. 19, by retracting the lead body 136. As such, sufficient deployment of the exterior anchors 165 means that the exterior anchors 165 abut the septal wall 220 during the retraction of the lead body 136 and do not re-enter the catheter 200. Thus, while greater expansion of the exterior anchors 165 is permissible, they should at least be expanded so that they are not retracted into the catheter 200.

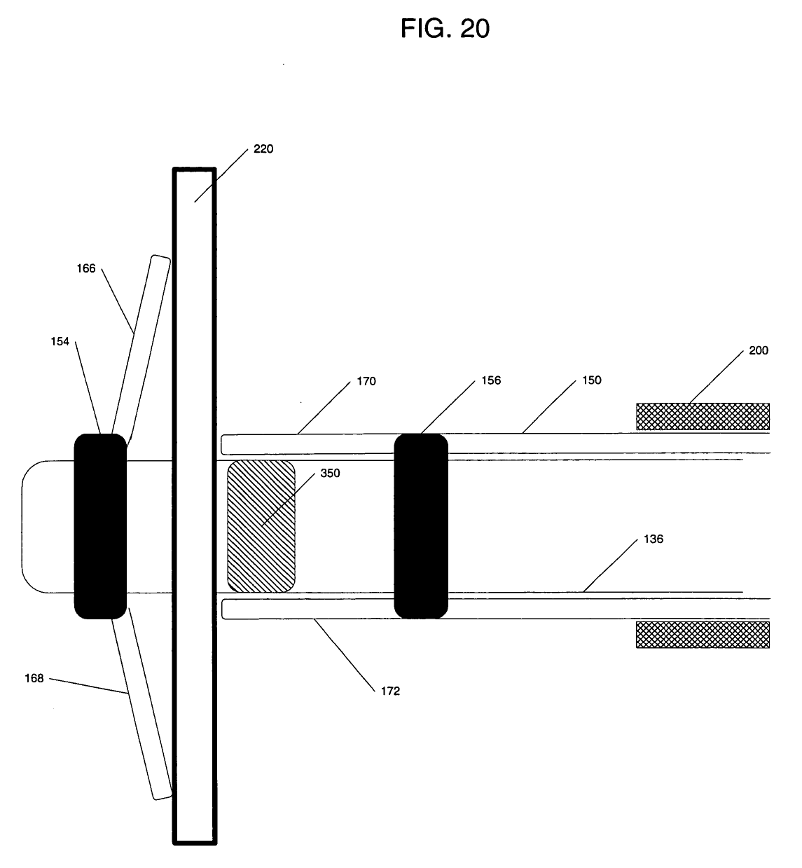

With the deployment balloon 350 deflated, and the lead body 136 retracted, as illustrated in FIG. 19, the exterior anchors 165 abut and are biased against the septal wall 220. The sheath 150 is advanced distally, relative to the lead body 136, so that the deployment balloon is proximate the distal ends of the interior anchors 175. Either as a separate step, or simultaneously, the catheter is retracted in a proximal direction, relative to the sheath 150 so that the interior anchors 175 are exposed within the right atrium 30 as illustrated in FIG. 20.

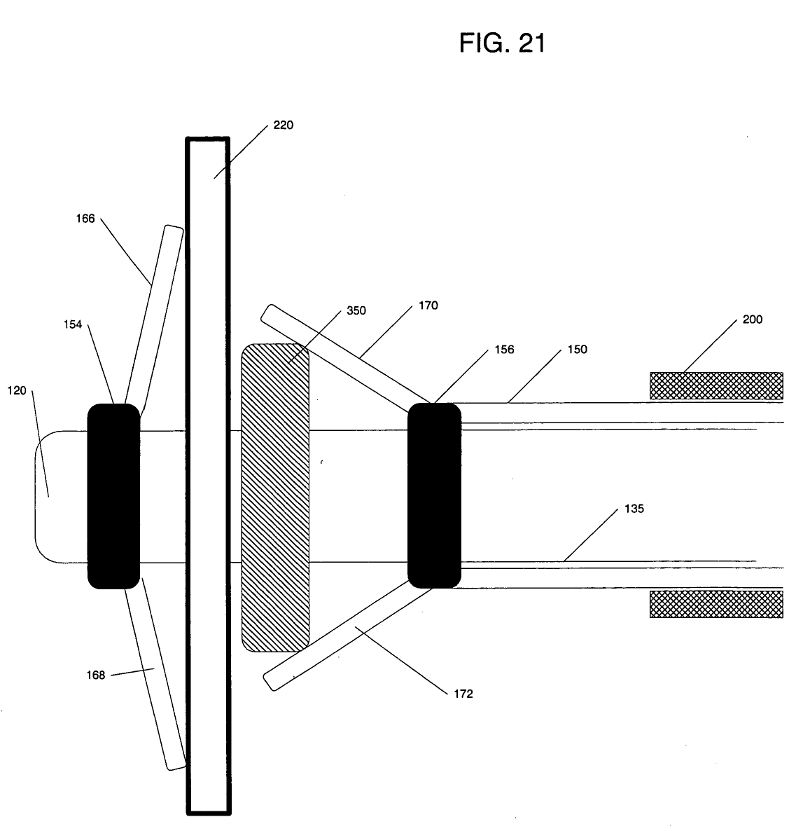

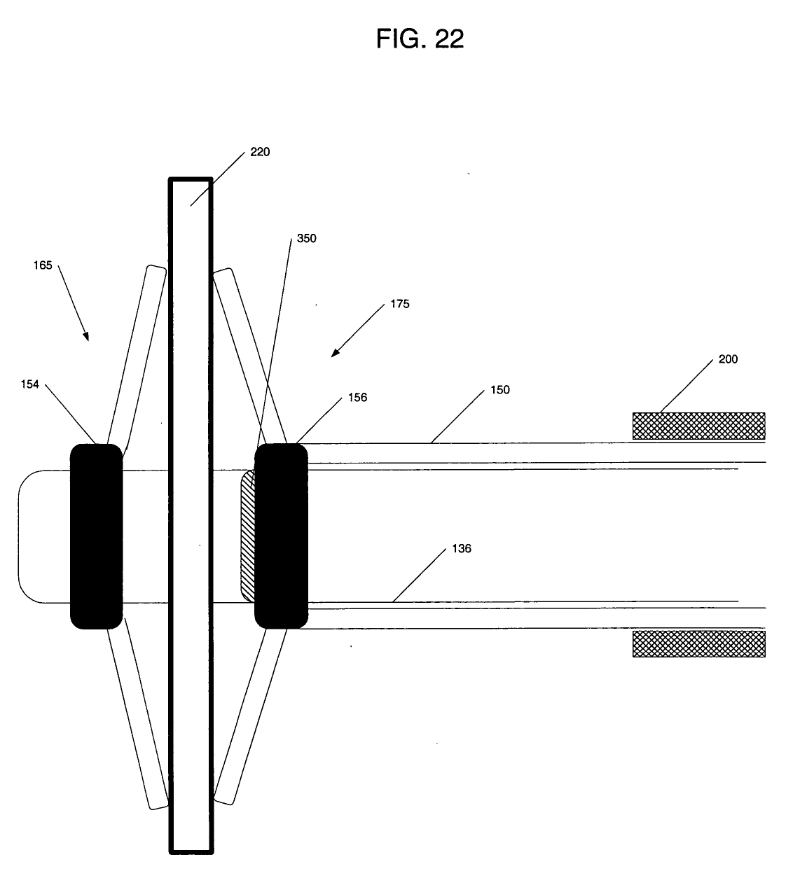

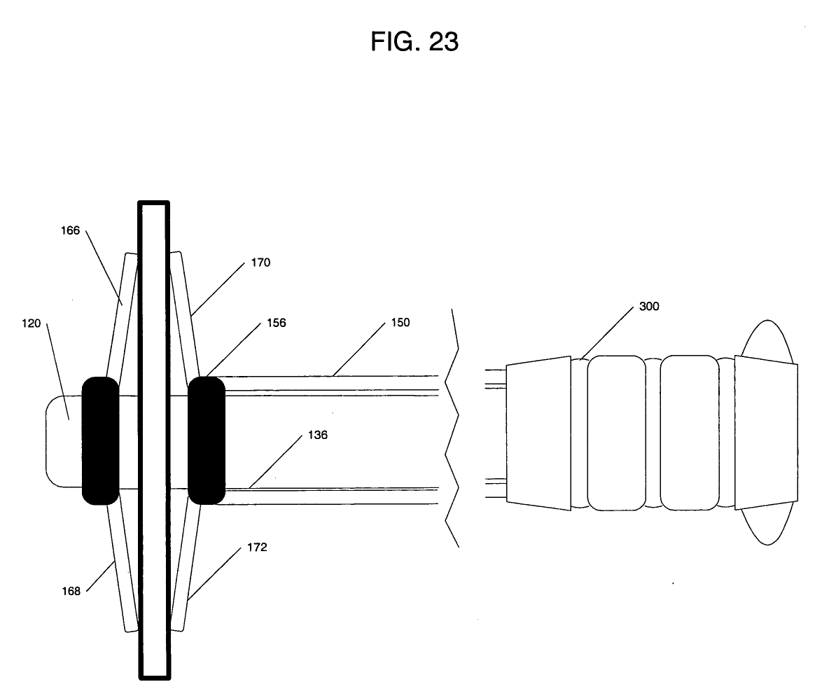

The deployment balloon 350 is expanded, causing the interior anchors 175 to pivot or flex relative to the proximal ring 156, as illustrated in FIG. 21. The sheath 150 is advanced distally after the deployment balloon 350 is deflated, which causes the interior anchors to expand further, as shown in FIG. 22. With the deployment balloon 350 deflated, the proximal ring 156 slides over the deployment balloon 350. Thus, the proximal ring 156 may serve as a shield or barrier for the deployment balloon 350 in some embodiments. Referring to FIG. 23, the position of the proximal ring 156 and sheath 150 are secured relative to the lead body 136 by the anchor sleeve 300. Thus, the exterior 165 and interior anchors 175 hold the pressure sensor 120 in position. As illustrated in FIG. 23, the proximal ring 156 completely covers the deployment balloon 350.

During implantation, if an issue arises the anchors may be retracted and the lead 100 removed and replaced. After implantation, should the need arise the anchors 165, 175 may be surgically cut and removed, leaving a hole in the septal wall 220. If a new lead 100 were not implanted, the hole would be surgically closed in the known way.

It should be appreciated that the deployment balloon 350 is not limited to an embodiment wherein the interior anchors are initially separated from the exterior anchors. That is, the deployment balloon 350 may be utilized with previous embodiments having break points 158. The deployment balloon 350 may by used to flex the break points 158 or sever them in another manner. Furthermore, the deployment balloon may be utilized to aid the expansion of either set of anchors after the break point has been severed. Thus, the deployment balloon may be utilized in embodiments of the lead 100, wherein the exterior anchors 165 are initially coupled with the interior anchors 175, as well as in embodiments wherein the exterior anchors are separate from the interior anchors prior to deployment.

As disclosed herein, a number of embodiments have been shown and described. These embodiments are not meant to be limiting and many variations are contemplated within the spirit and scope of the invention, as defined by the claim. Furthermore, particular elements illustrated and described with respect to a given embodiment are not limited to that embodiment and may be used in combination with or substituted into other embodiments.

We claim:

1. An implantable medical device (IMD) comprising:
an elongated lead body;
a pressure sensor disposed at a distal end of the lead body;
a first anchor member disposed proximate the pressure sensor and coupled with the lead body;
a second anchor member disposed proximate the pressure sensor, coupled with the lead body; and
a deployment balloon configured to selectively extend the first anchor member and to selectively extend the second anchor member by selective expansion of the deployment balloon.

2. The IMD of claim 1, further comprising:
a first support member slidably engaged with the lead body and forming the coupling of the first anchor member to the lead body; and
a second support member fixedly coupled with the lead body and forming the coupling of the second anchor member to the lead body.

3. The IMD of claim 2, further comprising an actuation member coupled with the first support member and operable from a proximal end of the lead body to effectuate linear movement of the first support member with respect to the lead body.

4. The IMD of claim 3, wherein the actuation member is a sheath at least partially surrounding the lead body and coupled with the first support member.

5. The IMD of claim 3, wherein the first and second support members are radio-opaque rings.

6. The IMD of claim 3, wherein engagement of the actuation member causes the first support member to move towards the second support member and select which anchor member is extendable by expansion of the deployment balloon.

7. An implantable medical device (IMD) comprising:
a lead body;
means for sensing pressure coupled with the lead body;
means for anchoring the pressure sensor to a substrate; and
means for identifying a location of predetermined portions of the implantable medical device using imaging.

8. The IMD of claim 7, further comprising means for selectively extending the means for anchoring.

9. The IMD of claim 8, further comprising means for positioning the means for selectively extending relative to the means for anchoring.

10. An implantable medical device comprising:
a lead body having a proximal end and a distal end;
a pressure sensor disposed proximate to the distal end of the lead body;
a sheath surrounding at least a portion of the lead body and accessible from the proximal end;
a first ring coupled with a distal end of the sheath so that the sheath and first ring are selectively and operably moveable relative to the lead body;
a second ring coupled to the lead body at a location distal to the first ring;
a first anchor member flexibly coupled with a distal side of the first ring;
a second anchor member flexibly coupled with a proximal side of the second ring; and
a deployment balloon forming a portion of the lead body proximate the distal end, wherein the deployment balloon is selectively expandable from the proximal end of the lead body to extend the first anchor member and to independently extend the second anchor member.

11. The IMD of claim 10, wherein the first and second ring are formed from a radio-opaque material.

12. The IMD of claim 10, further comprising an anchor sleeve disposed near the proximal end of the lead body to selectively lock the first ring with respect to the lead body.

13. The IMD of claim 10, further comprising a plurality of first anchor members and a plurality of second anchor members, wherein the first and second anchor members secure the lead body to a substrate with the first anchor member disposed on a first side of the substrate and the second anchor member disposed on a second side of the substrate, opposite to the first side.

14. The IMD of claim 10, wherein the first ring is selectively moveable over the deployment balloon after the second anchor is extended.

15. The IMD of claim 10, wherein the first ring is positioned over the deployment balloon after extension of the second anchor so that the first ring covers an entirety of the deployment balloon.

16. The IMD of claim 10, wherein the first anchor member is coupled to the second anchor member at a break point, wherein flexation of the break point separates the first anchor member from the second anchor member.

17. The IMD of claim 16, wherein expansion of the deployment balloon causes flexation of the break point.

18. The IMD of claim 16, wherein the break point is formed from a biodegradable material.

19. The IMD of claim 18, wherein the material is a sugar composite.

20. The IMD of claim 18, wherein the material is a gelatin.
